(19) United States
(12) Patent Application Publication
(10) Pub. No.: US 2022/0003754 A1
(43) Pub. Date: Jan. 6, 2022

(54) TWO DIMENSIONAL MATERIAL BASED PAPER MICROFLUIDIC DEVICE TO DETECT AND PREDICT ANALYTE CONCENTRATIONS IN MEDICAL AND NON-MEDICAL APPLICATIONS

(71) Applicant: Neil Mitra, Waterloo (CA)

(72) Inventor: Neil Mitra, Waterloo (CA)

(21) Appl. No.: 16/918,109

(22) Filed: Jul. 1, 2020

(51) Int. Cl. G01N 33/52; B01L 3/00; G01N 21/78; G01N 21/84; G06T 7/90; G16H 30/40

(52) U.S. Cl. CPC G01N 33/523; B01L 3/502707; G01N 21/78; G01N 21/8483; G01N 2021/752; G16H 30/40; B01L 2300/0864; B01L 2300/126; G06T 7/90

(57) ABSTRACT

The two-dimensional material (such as graphene, hBN) based Paper Microfluidic Device will detect various analytes, which can be related to disease conditions, to minimize guesswork for a common individual to recognize a certain analyte, and faster confirmation of analytes for professionals. It will enable to predict an increase of analyte concentrations through machine learning, be applicable in both medical and non-medical fields, have a refill enclosure, and a GPS activated app to contact nearby critical infrastructure in cases of medical applications. This will have the ability to detect analytes far faster than any traditional analyte detection systems in both medical and non-medical fields.

### FIELD OF INVENTION

The present invention relates generally to find a solution that includes the use of a two dimensional material in a paper microfluidic device in order to detect and predict various analytes which can be related to disease conditions, in order to minimize guesswork for a common individual to recognize a certain analytes, and faster confirmation of analytes for Professionals (for example in cases of disease condition analyte detection).

### BACKGROUND OF THE INVENTION

There are countless number of disease conditions that can affect a human being. Many conditions such as Myocardial Infarction, can be diagnosed in a hospital environment. However, such methods are only accessible in hospitals after a person has a Myocardial Infarction (or similar types of conditions). There are also methods which can show if a person has a disease condition (like Myocardial Infarction) using paper microfluidic devices, which have a long response time (around 4 hours) [1]. Disease condition detections are based on analytes such as biomarkers. This invention generally relates to detect and predict the concentration of any analyte. For example, be able to detect and predict disease conditions.

In this methodology, it is proposed a design for a two dimensional based Paper Microfluidic Device, which has the ability to quickly detect and also predict analyte concentrations. This will eliminate the guesswork involved in determining an analyte for a common individuals and have the ability to give fast readings for an analyte to Paramedics and Hospitals (in case of a disease condition) or for fast testing and screening of target molecule(s) at a very high accuracy.

The paper microfluidic device will have multiple analyte detection areas (for example, in disease conditions various biomarkers) by using color change. This will be done by using an Antigen-Antibody combination method for the analyte detection. For example, in the case of a Myocardial Infarction detection, biomarkers such as Troponin, CK-MB, IL-3, IL-4, and Myoglobin are used as the antibody analytes for the antigen binding in the analyte detection areas.

The fabrication of the paper microfluidic device will be done by using a standard inkjet printer to print the guidelines for the paper microfluidic device on Whatman Grade 4 paper. Then the printed device is put on a hot plate to create a hydrophobic barrier for laminar flow in the channels of the paper device.

The multiple analyte detection sections of the device which detects the presence of a certain analyte, has the given antibody analyte molecules dispensed in such sections beforehand.

The first design will include the use of a two dimensional material such as graphene as a filtration system for the analyte in order to filter out any solution material (for example blood in cases of medical applications) for faster detection [2]. The second design will include the use of a two dimensional material such as graphene to accelerate the analyte solution flow, by applying the two dimensional material (such as graphene) through the primary channel [3]. The third design will involve using a two dimensional material such as graphene both as a filter and a fast pathway for the analyte solution (for example to filter out biomarkers and accelerate blood flow in medical applications). The fourth design will involve using a two dimensional material such as graphene, sandwiched between two paper microfluidic devices and use the two dimensional material as an analyte solution filter. The fifth design will involve using a two dimensional material such as graphene, sandwiched between two paper microfluidic devices and use the two dimensional material to accelerate the analyte solution flow, by applying the two dimensional material (such as graphene) in the primary channel. The sixth design will involve using a two dimensional material such as graphene, sandwiched between two paper microfluidic devices and use the two dimensional material both as a filter and an accelerator for the analyte solution (for example to filter out biomarkers and accelerate blood flow in medical applications).

In order to test the effectiveness of the device, analyte concentrations with various concentration of specific analytes (in this case, of that of Myocardial Infarction) will be used (simulating cases of Myocardial Infarction detection) on the device to see the response time and how accurate the device is in terms of the color change detection, among many other detection factors which is described below.

In order to properly diagnose the presence of a specific analyte, the device will use three methods. The first using an image classification system using convolutional neural networks (CNN) in order to show current analyte concentration. In the case of medical applications, it predicts when a disease condition will occur. This will be possible by taking multiple pictures of the color change detection of the various concentrations of the analytes (in medical applications, the analytes are biomarkers). Then these images will be put through a convolutional neural network in order to classify the concentration levels of any given scenario of the given analyte solution. The paper microfluidic device will be underneath a camera, which is inside an enclosure apparatus. This camera will be able to take a picture of the color change of the paper microfluidic device in order to determine the presence of a certain analyte. The camera will be connected to an Arduino Microcontroller which would carry out the computational processes. A LCD display will also be connected to the Arduino Microcontroller to display the concentration level of the given analyte. In cases of medical applications, it will display if the person has the given disease condition (based on the analyte detection) or when the person will most likely have the disease condition (based on the analyte detection). The second method involves using a two dimensional material such as graphene as an electrical conductor at the areas where the color detection of the analyte takes place due to the binding event of the target molecule. Using the two dimensional material as an electrical conductor, the amount of analytes in the concentration can be precisely counted and be displayed on the LCD display [4][5]. The third method will combine both methods; the camera and CNN will conduct prediction methodology (in cases of medical applications), and the two dimensional material conduction method will accurately calculate the amount of biomarkers present.

In order to prove such paper microfluidic device is possible, a Graphene Oxide (GO) Integrated Paper Microfluidic Device (PMD) to detect and predict Myocardial Infarctions (MI) is developed and shown as a proof of concept. Here, paper microfluidics with integrated GO filter acts as a low cost rapid Point-of-care (POC) device. Such system has the ability to not only detect MI, but also predict the prognosis and increase of the associated biomarkers. The detection principle is based on colorimetric change due to antigen-antibody binding of the respective target cardiac biomarkers. An artificial intelligence-based application is developed to provide quantitative results from scanning the colorimetric change on the device as well as predicting the overall increase of the cardiac biomarkers. The test results could be transmitted to a hospital database for mobilizing paramedics to attend the individuals in need.

### SUMMARY OF THE INVENTION

One aspect of the invention provides a two dimensional material based Paper Microfluidic Device in order to filter and accelerate any solution, with or without analytes.

A further aspect of the invention provides a two dimensional material base Paper Microfluidic Device using analyte detection areas for Antigen-Antibody bindings to specific analytes.

A further aspect of the invention provides the two dimensional material based Paper Microfluidic Device to effectively detect an analyte in any solution.

A further aspect of the invention provides the two dimensional material based Paper Microfluidic Device to effectively predict how an analyte concentration will increase (such as biomarkers in the case of medical applications).

A further aspect of the invention provides the two dimensional material based Paper Microfluidic Device to use a Convolutional Neural Network for precise image classification for a specific analyte detection and prediction.

A further aspect of the invention provides the two dimensional material based Paper Microfluidic Device as an electrical conductor in order for precise count of analytes.

### BRIEF DESCRIPTION OF THE DRAWINGS

The forgoing and other aspects of the invention will become more apparent from the following description of specific embodiments thereof and the accompanying drawings which illustrate, by way of example only, the principle of invention. In the Drawings:

FIG. 1 shows the Paper Microfluidic Device without the fabricated two dimensional material. The five analyte detection regions are shown as an example. The actual embodiment can have multiple analyte detection regions.

FIG. 2 shows the two dimensional based Paper Microfluidic Device using graphene (as the two dimensional material) as an analyte solution filtration system.

FIG. 3 shows the two dimensional based Paper Microfluidic Device using the graphene (as the two dimensional material) as an accelerated analyte solution flow system.

FIG. 4 shows the two dimensional based Paper Microfluidic Device using the graphene (as the two dimensional material) both as an analyte solution filtration system and an accelerated analyte solution flow system.

FIG. 5 shows the two dimensional based Paper Microfluidic Device using the graphene (as the two dimensional material) sandwiched between two paper microfluidic devices and use the two dimensional material as an analyte solution filtration system.

FIG. 6 shows the two dimensional based Paper Microfluidic Device using the graphene (as the two dimensional material) sandwiched between two paper microfluidic devices and use the two dimensional material as an accelerated analyte solution flow system.

FIG. 7 shows the two dimensional based Paper Microfluidic Device using the graphene (as the two dimensional material) sandwiched between two paper microfluidic devices and use the two dimensional material both as an analyte solution filtration system and an accelerated analyte solution flow system.

FIG. 8 shows the two dimensional based Paper Microfluidic Device underneath a camera which is connected to an LCD display via an Arduino Uno microcontroller all inside an enclosure.

FIG. 9 shows the two dimensional based Paper Microfluidic Device using the graphene (as the two dimensional material) as an electrical conductor in the analyte detection areas connected to an LCD display and Arduino Uno microcontroller all inside an enclosure.

FIG. 10 shows the two dimensional based Paper Microfluidic Device using the graphene (as the two dimensional material) as an electrical conductor in the analyte detection areas, underneath a camera which is connected to an LCD display via an Arduino Uno microcontroller all inside an enclosure.

FIG. 11 shows the two dimensional based Paper Microfluidic Device enclosure being paired with an Android GPS application to communicate to nearby critical infrastructures.

FIG. 12 shows the fabrication workflow for PMD.

FIG. 13 shows the method to generate the calibration curve for the PMD.

FIG. 14 shows the method to optimize the GO filter (in terms of its quantity and length) and the amount of Methylene Blue Dye (MBD) sample.

FIG. 15 shows the method to verify the working principle of the GO filter in terms of effectively retaining Polystyrene Beads (PB) and allowing MBD to flow to the detection zones.

FIG. 16 shows the method for DBP for cTnI detection.

FIG. 17 shows the formula used to calculate the retention coefficient of the GO filter.

FIG. 18 shows the recorded time of different amount of MBD flow through different amount of deposited GO.

FIG. 19 shows the calibration curve for different MBD concentrations without GO filter.

FIG. 20 shows the recorded time taken for different amount of MBD to flow through PMD without deposited GO.

FIG. 21 shows the recorded time of different amount of MBD flow through different GO filter length with varying amount of GO.

FIG. 22 shows the variation of retention coefficient with different GO amount deposited. For each GO amount, the filter length was varied between 0.5 cm-1.5 cm.

FIG. 23 shows the microscope images of (a) the paper before GO filter; (b) the GO filter; (c) the paper after the GO filter during the flow of 125 μl simulate blood sample containing 55% MBD and 45% PB.

FIG. 24 shows the image of NC membrane for DBP for different concentration of cTnI.

FIG. 25 shows the classification accuracy of CNN based on the color images of NC Membrane (see FIG. 24) for different concentration of cTnI.

FIG. 26 shows the accuracy of the algorithm to predict the rate of increase of the cTnI concentration over time.

FIG. 27 shows the individual time taken for the prediction algorithm, CNN, and the combined CNN-prediction algorithm.

### DETAILED DESCRIPTION OF AN EMBODIMENT

The description which follows, and the embodiments describe therein, are provided by way of illustration of an example, or examples, of particular embodiments of the principles of the present invention. These examples are provided for the purpose of explanation, and not limitation, of those principles and of the invention.

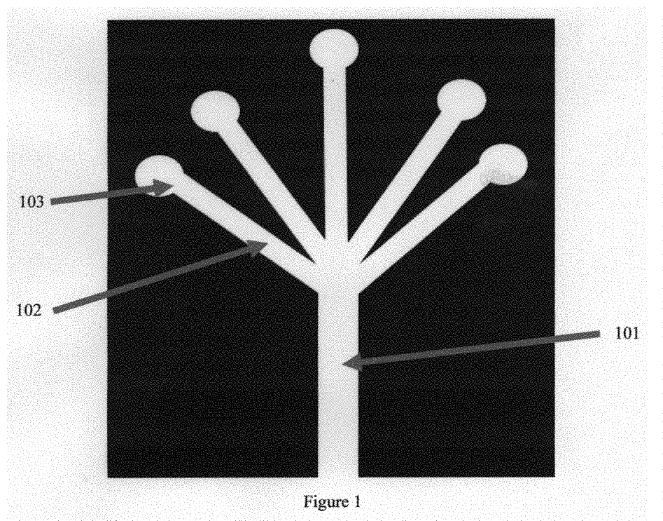

The original base level Paper Microfluidic Device with no two dimensional material or antibody integration in FIG. 1. As shown in FIG. 1, the device consists of a primary channel 101, secondary channels leading to the analyte binding areas 102, and the antigen-antibody analyte binding locations 103.

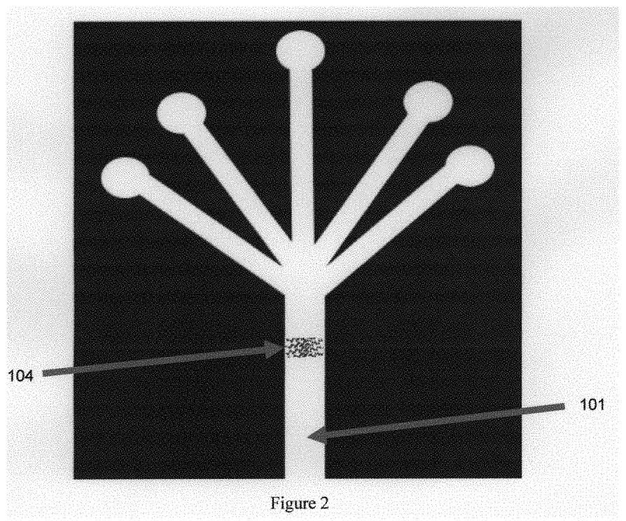

The Paper Microfluidic Device has two dimensional material integration, in the case shown the two dimensional material is graphene, in FIG. 2. The graphene 104 used in the Paper Microfluidic Device is used as an analyte filtration system placed in the primary channel 101. This is achieved by creating a larger hexagonal lattice of the two dimensional material (in this case graphene) to let target molecules to go through, while the buffer solution is hindered.

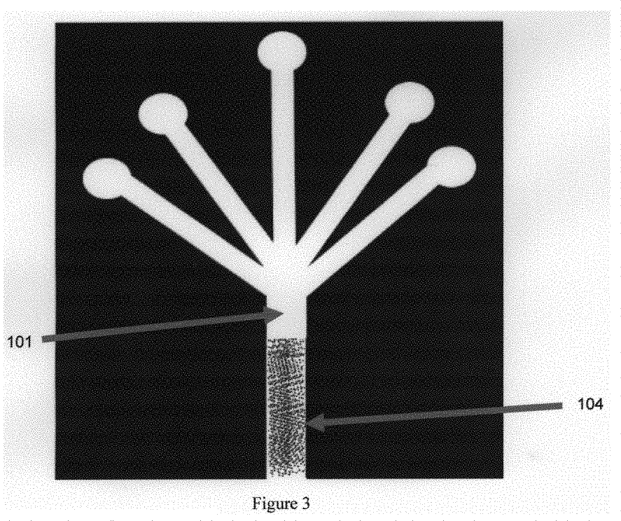

The Paper Microfluidic Device has two dimensional material integration, in the case shown the two dimensional material is graphene, in FIG. 3. The graphene 104 used in the Paper Microfluidic device is used as an accelerated analyte solution flow system placed all through the primary channel 101. This analyte solution accelerator flow system is achieved by using the two dimensional material (in this case graphene) as the hydrophilic substance to accelerate the analyte solution (buffer solution) movement throughout the primary channel 101.

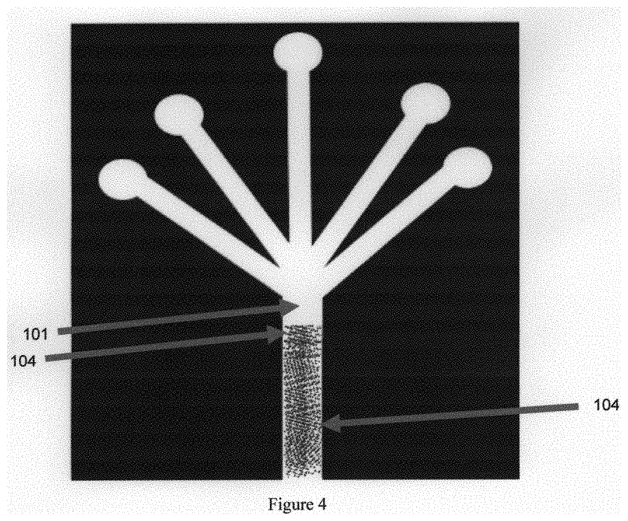

The Paper Microfluidic Device has two dimensional material integration, in the case shown the two dimensional material is graphene, in FIG. 4. The graphene 104 used in the Paper Microfluidic device is used both as a target molecule filtration system placed in the primary channel 101 and an accelerated analyte solution (buffer solution) flow system placed all throughout the primary channel 101.

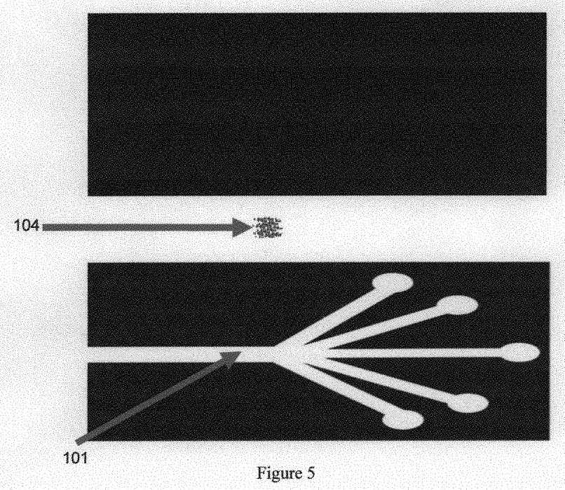

In FIG. 5, the two dimensional material, in the case shown the two dimensional material is graphene, is sandwiched between two Paper Microfluidic Devices. The graphene 104 used between the two Paper Microfluidic devices is used as an analyte (target molecule) filtration system placed in the primary channel 101. This is achieved by creating a larger hexagonal lattice of the two dimensional material (in this case graphene) to let analytes (given target molecules) to go through, while the analyte solution (buffer solution) is hindered.

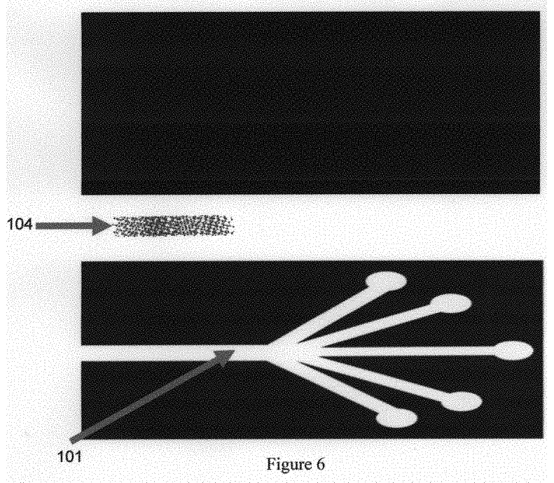

In FIG. 6, the two dimensional material, in the case shown the two dimensional material is graphene, is sandwiched between two Paper Microfluidic Devices. The graphene 104 used in the Paper Microfluidic device is used as an accelerated analyte solution flow system placed all through the primary channel 101. This analyte solution (buffer solution) accelerator flow system is achieved by using the two dimensional material (in this case graphene) as the hydrophilic substance to accelerate the analyte solution (buffer solution) movement throughout the primary channel 101.

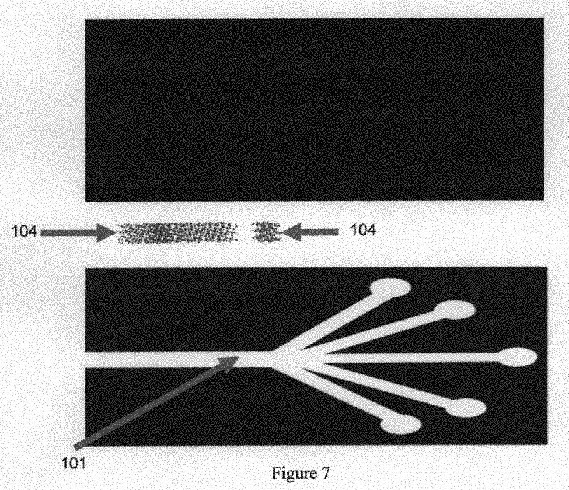

In FIG. 7, the two dimensional material, in the case shown the two dimensional material is graphene, is sandwiched between two Paper Microfluidic Devices. The graphene 104 used in the Paper Microfluidic device is used both as an analyte (target molecule) filtration system placed in the primary channel 101 and an accelerated analyte solution (buffer solution) flow system placed all throughout the primary channel 101.

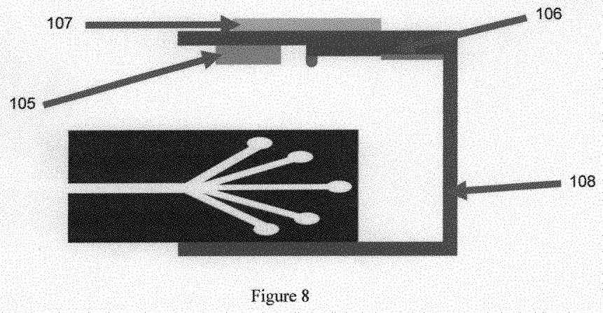

In FIG. 8, the two dimensional based Paper Microfluidic device can be any of the different designs shown through FIG. 1 to FIG. 7. The two dimensional based Paper Microfluidic device is placed underneath a two megapixel Arduino compatible camera 105. The camera 105 is then connected to an Arduino Uno 106 which is connected to an LCD display 107, all enclosed in an enclosure system 108 so that the used two dimensional based Paper Microfluidic Devices can be replaced by new two dimensional based Paper Microfluidic Devices.

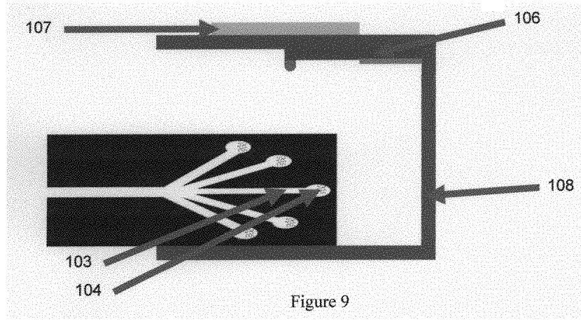

In FIG. 9, the two dimensional based Paper Microfluidic device can be any of the different designs shown through FIG. 1 to FIG. 7. In this case, additional two dimensional material which has conductive properties (in this case graphene 104) is added to the analyte detection areas 103. This allows the graphene to give a precise count of analytes (target molecules) associated within the buffer solution or analyte solution (in case of medical applications, biomarkers in blood). The Arduino Uno 106 is connected to the two dimensional material (in this case graphene 104) in the analyte detection area 103 to perform such count of analytes. The Arduino Uno 106 is also connected to the LCD display 107, and is enclosed underneath an enclosure system 108 so that the used two dimensional based Paper Microfluidic Devices can be replaced by new two dimensional based Paper Microfluidic Devices.

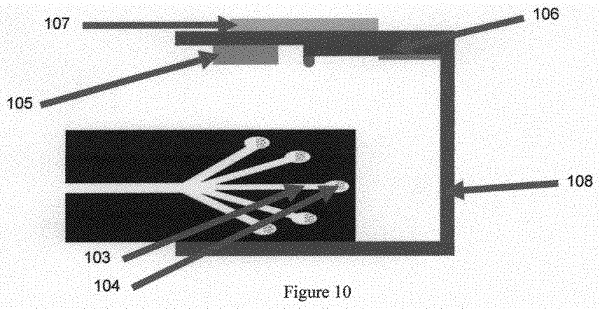

In FIG. 10, the two dimensional based Paper Microfluidic device can be any of the different designs shown through FIG. 1 to FIG. 7. In this case, additional two dimensional material which has conductive properties (in this case graphene 104) is added to the analyte detection areas 103. This allows the graphene to give a precise count of analytes associated within the solution (in case of medical applications, biomarkers in blood). The Arduino Uno 106 is connected to the two dimensional material (in this case graphene 104) in the analyte detection area 103 to perform such count of analytes (target molecules). The Arduino Uno 106 is also connected to the LCD display 107, and a two megapixel Arduino compatible camera 105 is enclosed underneath an enclosure system 108 so that the used two dimensional based Paper Microfluidic Devices can be replaced by new two dimensional based Paper Microfluidic Devices.

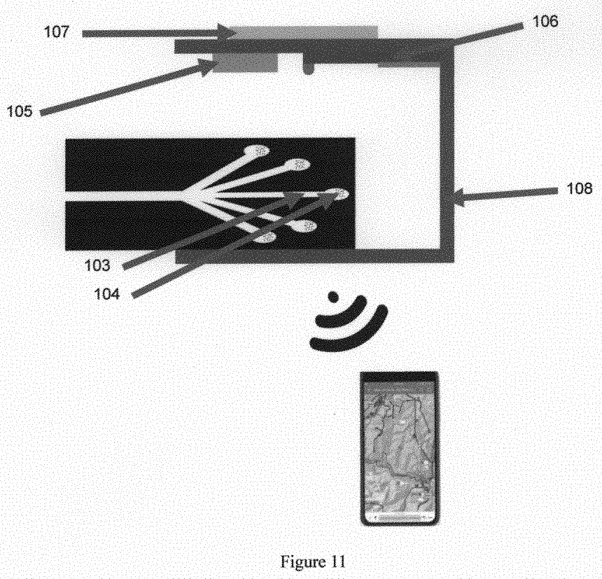

In FIG. 11, the two dimensional material based Paper Microfluidic device enclosure system 108 shown in FIG. 8 to FIG. 10, can be paired with a GPS enable Android APP in order to communicate quickly to nearby hospitals, in the case where a disease condition can immediately worsen (which is predicted).

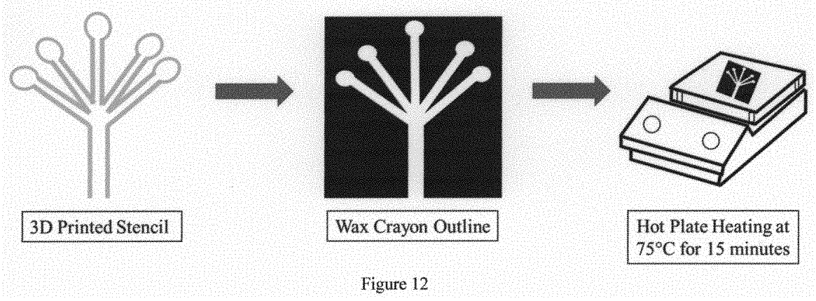

In FIG. 12, to fabricate the PMD, a 3D printed stencil was made using Ender 3 Pro Printer. A wax crayon was used to trace around the printed stencil on the Whatman Grade 4 filter paper to generate two specific detection zones. After the design was laid out, the PMD was placed on a hot plate for 15 minutes at 75° C. to create a hydrophobic barrier along the crayon outline. This would allow for laminar flow of reagents on the PMD.

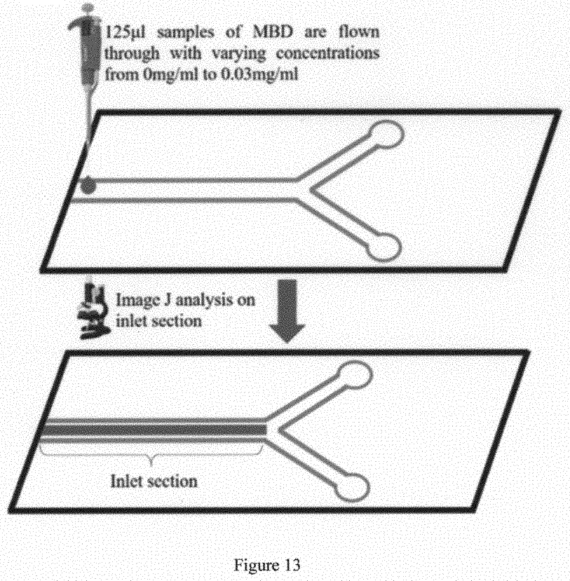

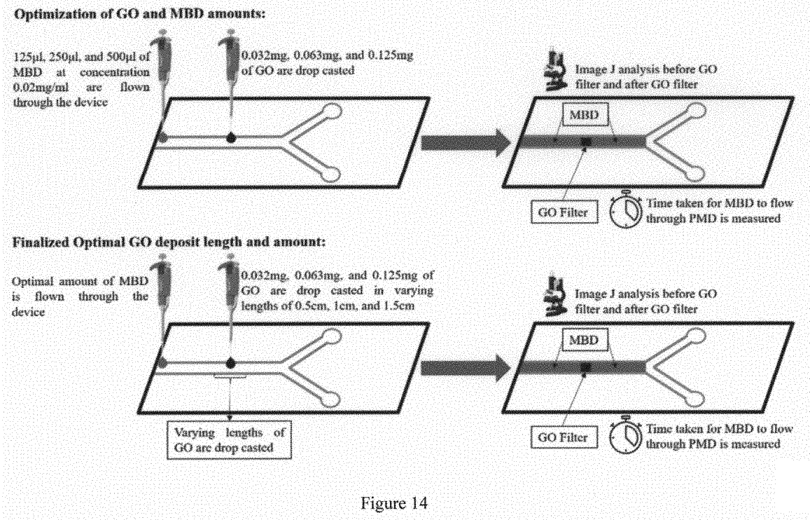

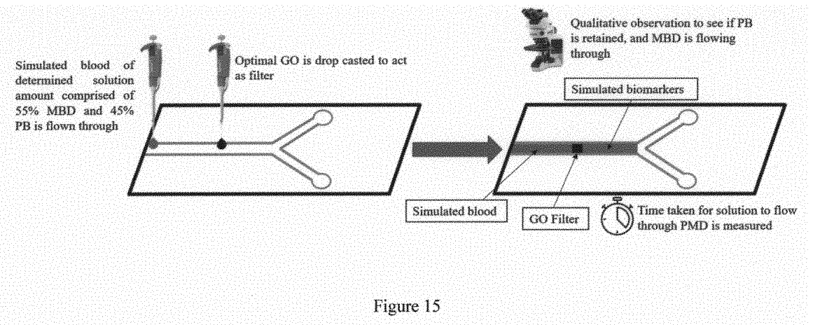

In FIGS. 13 through 15, MBD solution is used to simulate cardiac biomarkers and PB is used to simulate predominant blood constituents (Red Blood Cells and White Blood Cells). The optimized GO filter should retain most of the PB and only allow MBD to flow through. This technique will allow a faster antibody-antigen binding in the detection zones. First, a calibration curve for MBD is conducted using Image J RGB blue value color analysis. Five PMDs were used where 125 μl of MBD were flown through the PMD at varying concentrations from 0 mg/ml to 0.03 mg/ml. Successive images of the PMD were taken using the cell phone camera and RGB blue value analysis is performed on the inlet section of the PMD, as shown in FIG. 13. These experiments also provide the control test results as the flow through the PMD is conducted without having any GO filter. FIG. 14 shows how the optimal GO filter is tested and fabricated onto the PMD. To determine the optimal amount of MBD to flow through the PMD; 0.032 mg, 0.063 mg, and 0.125 mg of GO were drop casted onto the inlet section of the PMD for a fixed length of 0.5 cm. Thereafter, 125 μl, 250 μl, and 500 μl of MBD at concentration 0.02 mg/ml (to simulate concentrated cardiac biomarkers) are flown through the device. The RGB value before the GO filter and after the GO filter, as well as the time taken for the MBD to flow through, would determine the optimal amount of GO and MBD to use. Next, 0.032 mg, 0.063 mg, and 0.125 mg of GO were drop casted in varying lengths from 0.5 cm, 1.0 cm, and 1.5 cm on the PMD and the optimal MBD sample amount is flown through the device. The RGB value before the GO filter and after the GO filter, as well as the time taken for the MBD to flow through, would finalize the optimal amount and deposit length of GO to act as the most optimal filter. FIG. 15 demonstrates how the PB and MBD are used together to act as simulated blood to test the GO filter. A solution of a fixed amount (determined through the method described earlier) would contain 55% MBD and 45% PB to best simulate a human blood sample. This sample is flown through the PMD and qualitative results are carried out using an Olympus BX53M Upright Microscope and Image J analysis. The time taken for this simulated blood sample to flow through the GO filter integrated PMD is compared to the control tests, described earlier. A successful PMD would show that the PB is blocked by the GO filter allowing the flow of MBD only.

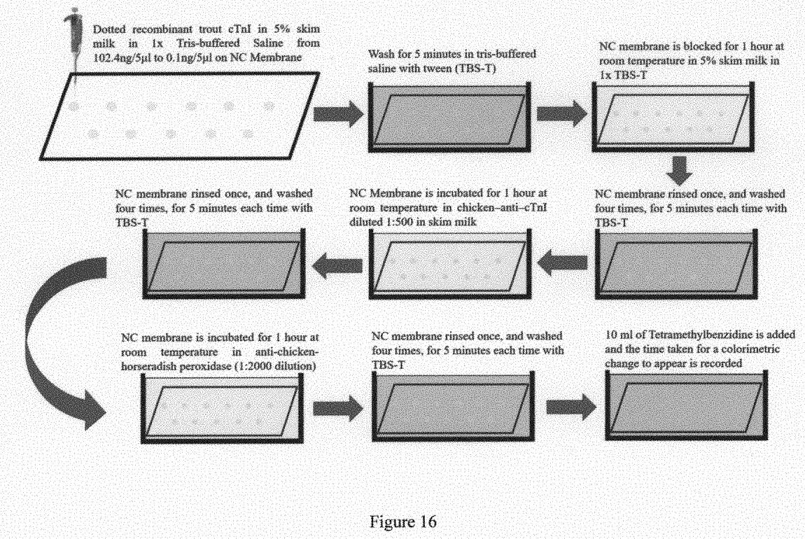

In FIG. 16, is shown how cTnI was detected using Dot Blot Protocol. First, serial dilutions of recombinant trout cTnI in 5% skim milk (within 1×Tris-buffered Saline) were prepared from 102.4 ng/5 μl to 0.1 ng/5 μl, as well as a negative control (0.0 ng/5 μl). The samples are then dotted onto a nitrocellulose (NC) membrane. Next, the NC Membrane is washed for 5 minutes in tris-buffered saline with tween (TBS-T). The NC membrane is then blocked for 1 hour at room temperature in 5% skim milk (within 1×TBS-T). The NC membrane is then rinsed once, and washed four times, for 5 minutes each time with TBS-T. Next, the NC Membrane is incubated for 1 hour at room temperature (25° C.) in chicken-anti-cTnI diluted 1:500 in skim milk. Thereafter, a similar washing and rinsing step is followed. Then, the NC membrane is incubated for 1 hour at room temperature in anti-chicken-horseradish peroxidase (1:2000 dilution) followed by washing and rinsing steps. Next, 10 ml of Tetramethylbenzidine is added and the time taken for a colorimetric change to appear is recorded.

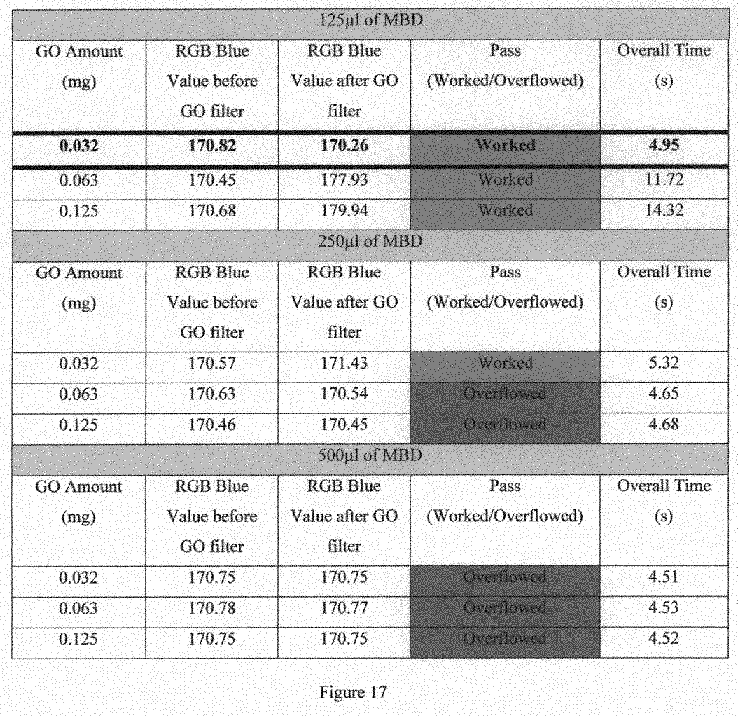

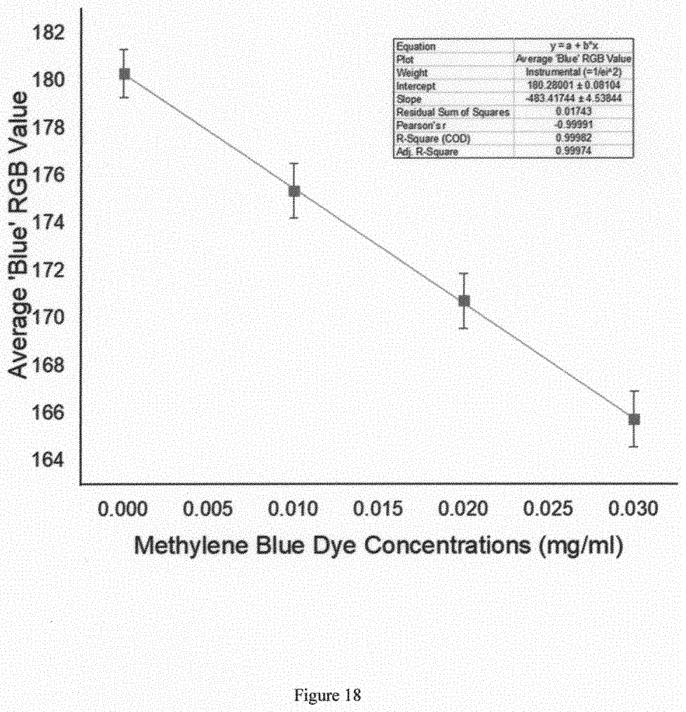

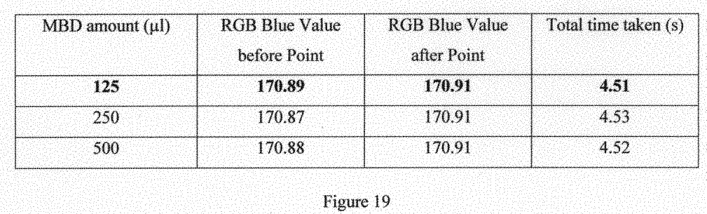

In FIGS. 17 to 19, one of the key components for a rapid POC device is the amount of sample required to complete a reliable detection. In this case, a successful sample amount is one that can be filtered properly through the GO filter. As shown in FIG. 17, for certain combination of the GO and MBD amounts, the device failed to operate. It is to be noted that the increase in the value of RGB blue after the GO filter in the inlet section of the PMD corresponds to the lower concentration of MBD, i.e., the GO is blocking the majority of MBD (as shown in FIG. 18). This needs to be avoided for the optimal operation of the GO filter. Therefore, from the FIG. 17, it is apparent that for the given range of GO and MBD used in this study, the optimal amount of GO to be deposited to act as a filter is 0.032 mg for 125 μl of MBD. Controlled tests for the paper microfluidics without the GO filter were also conducted using different amounts of MBD and measuring the time taken for MBD to flow through the inlet section. As shown in FIG. 19, the least time taken for MBD was 4.51 seconds for 125/11 of MBD. This is comparable with the time taken for MBD to flow in the actual PMD with GO filter (˜4.95 seconds as shown in FIG. 17).

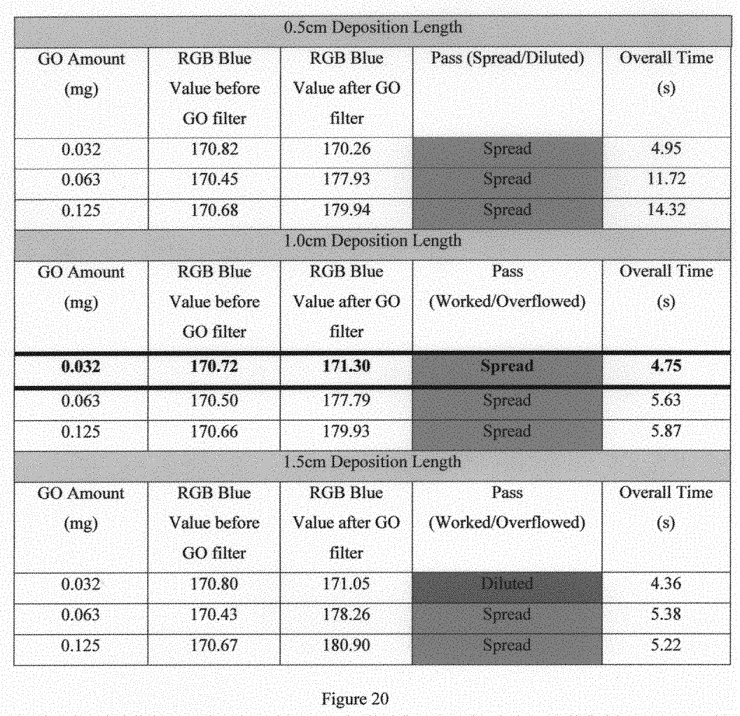

In FIGS. 17 and 20, the next component that is investigated is the effect of the distribution of GO over a certain length. From the previous experiment, a 1250 MBD sample is established to be the most optimal sample to flow through the PMD. However, the length of deposition is a key factor in determining how fast the MBD can pass through. Therefore, study is conducted where the length of the GO filter deposited is varied between 0.5 cm to 1.5 cm. It is to be noted that the results shown in FIG. 17 were also for 0.5 cm of GO filter length. For a given deposited length, the amount of GO was varied between 0.032 mg to 0.125 mg. For a small amount of GO deposited over a longer length, the GO filter fails (see FIG. 20). As shown in FIG. 20, a 0.032 mg of GO deposition spread across a length of 1 cm allows for the fastest filtration time of 4.75 seconds.

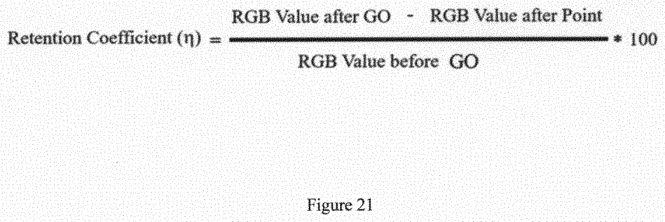

In FIG. 21, one of the key parameters that quantified the efficacy of the GO filter is to define its retention coefficient (η). It is defined as the amount of MBD that the GO filter allows to pass through normalized with the initial amount of MBD before it enters the GO filter. The amount of MBD that flows through can be found by taking the difference between the RGB blue value after the GO filter (third column in FIGS. 17 and 20) and the RGB blue value of the control test over the same length (third column in FIG. 19) of the paper microfluidics. The initial amount of MBD is determined by recording the RGB blue value before GO filter (second column in FIGS. 17 and 20). Hence, η can be calculated using the equation in FIG. 21.

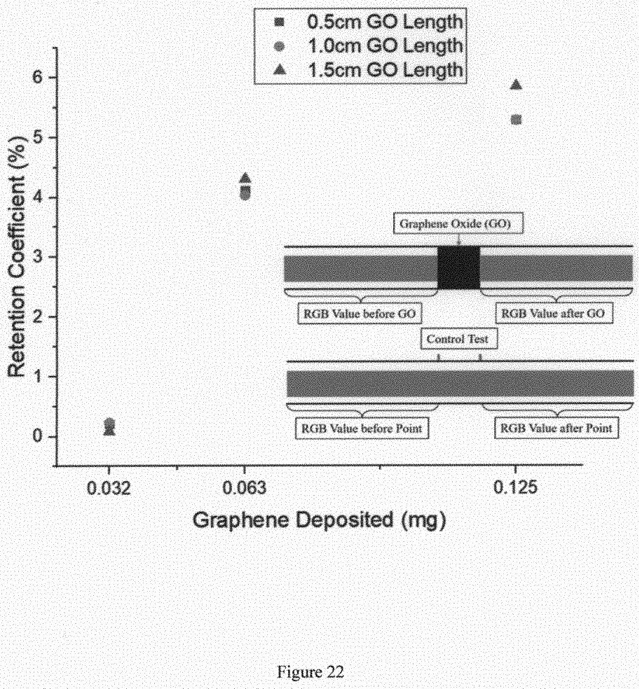

In FIG. 22, it is shown the variation of the retention coefficient, converted into percentage, for different amount of GO deposited and their corresponding lengths. In general, as expected, η increases with the increase in the amount of GO deposited, since higher amount of GO will tend to block most of the MBD sample. For a given GO deposited amount, beyond 0.032 mg, it is found that as the GO length increases (particularly in case of 1.5 cm), the retention coefficient increases. This is because over a longer length of GO filter, there will be additional resistance to the flow of MBD, which in turn increases the retention of MBD. As shown in FIG. 22, the lowest value of η (˜0.2%) is obtained with 0.032 mg of GO deposited for both 0.5 cm and 1.0 cm lengths. However, as shown in FIG. 20, for 0.032 mg of GO deposited, the fastest response is obtained for 1.0 cm length. Hence, it re-establishes that for the given choice of parameters, 0.032 mg of GO deposited over a filter length of 1.0 cm will ensure the optimal performance of PMD.

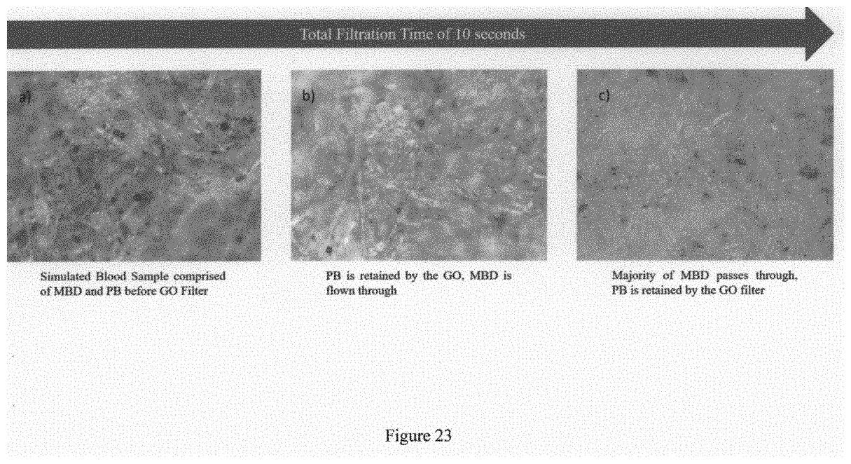

In FIG. 23, actual tests were conducted on PMD using 125 μl of simulated blood sample with the optimal load of the GO filter. The choice of the sample volume was dictated by the earlier study where the optimal amount of MBD was established to be 125 μl for the flow of samples on the PMD. FIG. 23 shows the microscope images of three different section of the inlet section of the PMD. It clearly shows that the used GO filter configuration is able to successfully PB and allow MBD to flow through within 10 seconds.

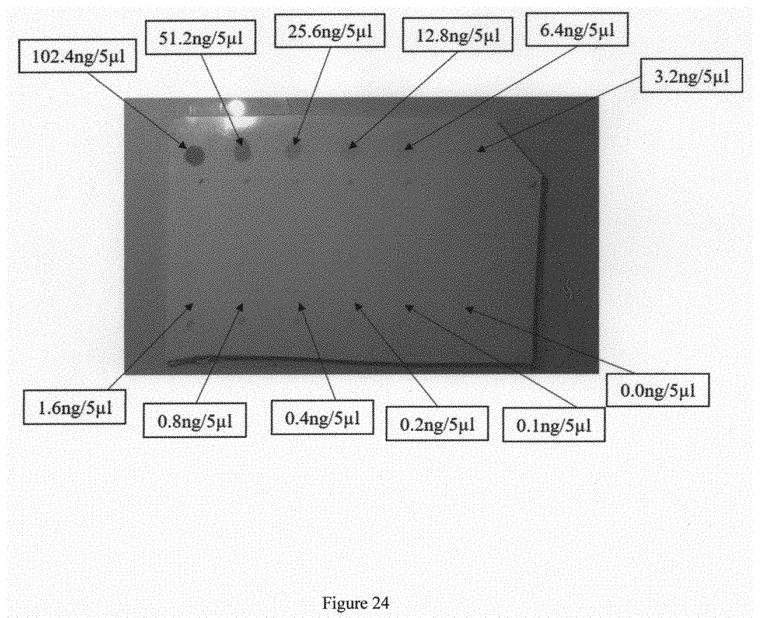

In FIG. 24, the development of a rapid POC would require an establishment of an efficient antigen-antibody binding that would result in a pronounced color change in the detection zones on the PMD. To establish this antigen-antibody binding, an ex situ Dot Blot Protocol is carried out on nitrocellulose membrane. FIG. 24 shows the color change on NC membrane for varying concentration (0.0 ng/5 μl-102.4 ng/5 μl) of cTnI. It is found that the color change occurs within 90 seconds for the concentration range of 6.4 ng/5 μl to 102.4 ng/5 μl. Therefore, this successful antigen-antibody binding assay can be readily implemented in the detection zones of the PMD at a later stage for successful deployment of this POC device.

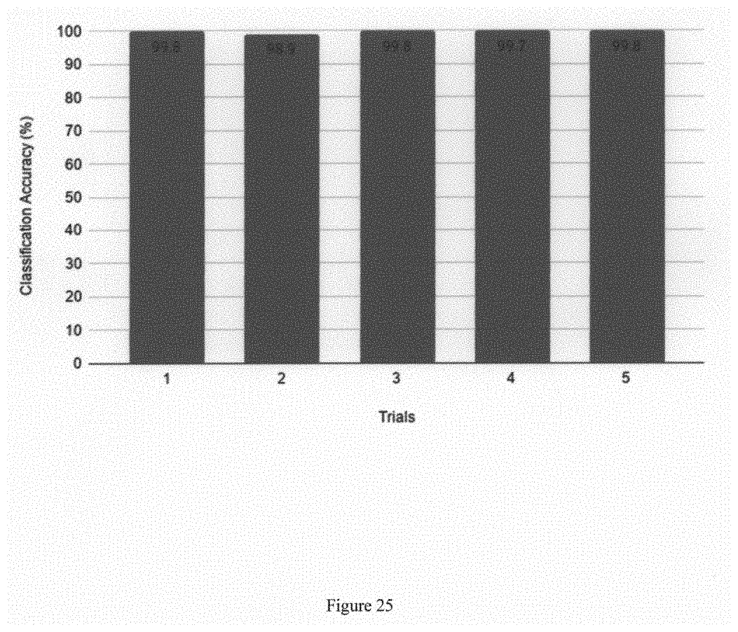

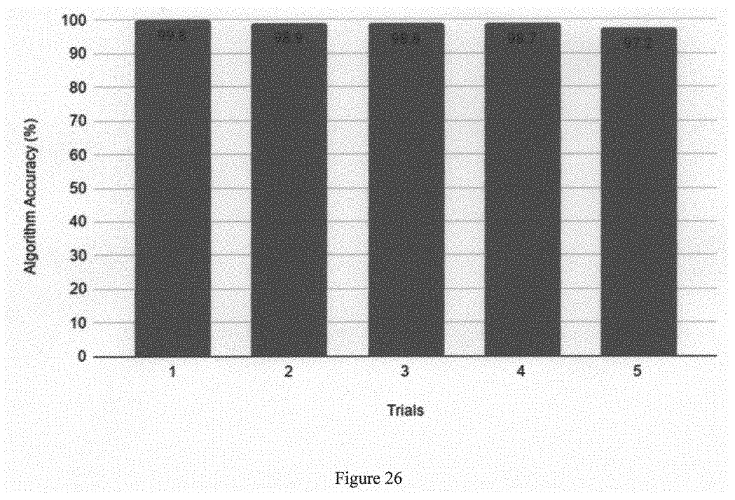

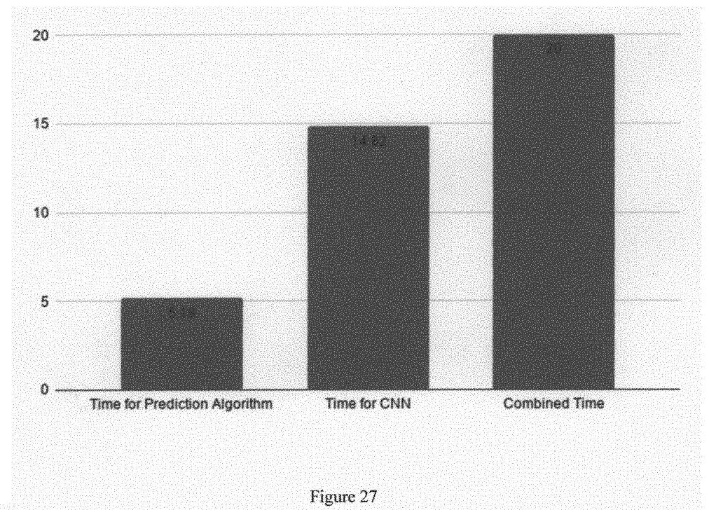

In FIGS. 25 to 27, when the POC is deployed in actual field testing, the blood samples collected will have unknown concentration of cTnI. Therefore, a predictive tool is also needed in conjunction with an efficient detection system to quantify the unknown amount of biomarkers in human blood samples. To quantify the amount of cTnI present, a CNN is made to properly classify the colorimetric change to the respective concentrations. To predict the increase of biomarkers, a prediction algorithm is developed. As shown in FIG. 25, the CNN has an average accuracy of 99.6% to quantify the cTnI concentration on NC membrane, used earlier for Dot Blot Protocol. As shown in FIG. 26, the prediction algorithm has an average accuracy of 98.7% to predict the linear increase of biomarker concentration in 5 hours depending on the quantified cTnI concentration by the CNN. The overall time for both the CNN and prediction algorithm to complete successful detection and prediction of cTnI is 20 seconds as shown in FIG. 27. A successful implementation of the integrated approach of combining detection with machine learning would enable the fastest way to detect and predict unknown concentration of cardiac biomarkers, which is currently not available. In this particular studied case, it takes 10 seconds for the GO filter to successfully filter simulated blood, 90 seconds for colorimetric antibody-antigen binding detection, and 20 seconds for the CNN and the predictive algorithm to provide result. The entire work flow can be now achieved within 2 minutes, which is a remarkable achievement in terms of the development of this novel POC. It is to be noted that even though the antigen-antibody binding event, which takes 90 seconds to occur, is conducted on NC membrane, rather on the actual PMD, it is expected that similar porous nature of the paper substrate with the NC membrane along with enhanced capillary transport that can be achieved for paper, might further reduce the detection time in the actual PMD.

### REFERENCES

1. Lim, W. Y., Thevarajah, T. M., Goh, B. T., & Khor, S. M. (2019). Paper microfluidic device for early diagnosis and prognosis of acute myocardial infarction via quantitative multiplex cardiac biomarker detection. Biosensors and Bioelectronics, 128, 176-185.
2. Cohen-Tanugi, D., & Grossman, J. C. (2012). Water desalination across nanoporous graphene. Nano Letters, 12(7), 3602-3608.
3. Huang, H., Song, Z., Wei, N., Shi, L., Mao, Y., Ying, Y., . . . & Peng, X. (2013). Ultrafast viscous water flow through nanostrand-channelled graphene oxide membranes. Nature Communications, 4, 2979.
4. Schedin, F., Geim, A. K., Morozov, S. V., Hill, E. W., Blake, P., Katsnelson, M. I., & Novoselov, K. S. (2007). Detection of individual gas molecules adsorbed on graphene. Nature Materials, 6(9), 652.
5. Shao, Y., Wang, J., Wu, H., Liu, J., Aksay, I. A., & Lin, Y. (2010). Graphene based electrochemical sensors and biosensors: a review. Electroanalysis, 22(10), 1027-1036.

We claim:

1. The use of any two dimensional material in the application of a Paper Microfluidic Device to detect and predict any concentration of target molecule analytes in order to decrease detection time and minimize self-detection guesswork for an individual, via combining the use of two dimensional material with paper microfluidics along with the use of the convolutional neural network for image classifier to detect and predict analyte concentration.

2. The Application of using a Camera or any type of reader and LCD connection to read colour change in any two dimensional material based Paper Microfluidic Device in order for detection and prediction for any type of target molecule analyte.

3. An APP that will send GPS coordinates of the device to the nearest hospital, in the case of a disease condition rapidly deteriorating.

4. The application of claim 1, wherein any analyte can be used for detection in both medical and non medical applications.

5. The application of claim 1, wherein any two dimensional material can be used anywhere on the two dimensional material based Paper Microfluidic device to accelerate any given analyte buffer solution.

6. The application of claim 1, wherein any two dimensional material can be used anywhere on the two dimensional material based Paper Microfluidic device to filter any specific target molecule within or without any analyte buffer solution.

7. The application of claim 1, wherein any two dimensional material can be used anywhere on the two dimensional material based Paper Microfluidic device to accurately count the number of analyte target molecules within or without any analyte buffer solution.

8. The application of claim 1, wherein any two dimensional material can be used anywhere on the two dimensional material based Paper Microfluidic device to simultaneously accelerate any given analyte buffer solution and filter any specific target molecule within or without any analyte buffer solution.

9. The application of claim 1, wherein any two dimensional material can be used anywhere on the two dimensional material based Paper Microfluidic Device to simultaneously accelerate any given analyte buffer solution, filter any specific target molecule within or without any analyte buffer solution, and accurately count the number of analyte target molecules within or without any analyte buffer solution.

10. The apparatus of claim 2, wherein an application of any image reader connected to any given microcontroller has the ability to correctly detect and predict any analyte target molecule within any part of the two dimensional material based Paper Microfluidic Device.

11. The apparatus of claim 2, wherein an application of any image reader connected to any given microcontroller has the ability to correctly detect and predict any analyte target molecule within any part of the two dimensional material based Paper Microfluidic Device, and display the results on any display screen.

12. The apparatus of claim 2, wherein an application of any image reader connected to any given microcontroller has the ability to correctly detect and predict any analyte target molecule within any part of the two dimensional material based Paper Microfluidic Device, display the results on any display screen as well as display the precise count of the analyte target molecules via any two dimensional material placed anywhere on the two dimensional material based Paper Microfluidic Device.

13. The apparatus of claim 3, wherein an application of any smartphone device can be connected to the enclosure of the two dimensional based Paper Microfluidic Device in order to contact nearby Hospitals and pinpoint location in cases of analyte target molecule detections in medical applications (if disease conditions worsen or are at very high levels).

14. The apparatus of claim 1, wherein an application of any design of the Paper Microfluidic Device can be used for the integration of a two dimensional material.

15. The apparatus of claim 1, wherein an application of any designed enclosure for all stated detection methods can be used.
